(19) United States
(12) Patent Application Publication
(10) Pub. No.: US 2021/0309952 A1
(43) Pub. Date: Oct. 7, 2021

(54) SYSTEMS FOR CELL PLATE CO-CULTURE

(71) Applicant: Board of Supervisors of Louisiana State University and Agricultural and Mechanical College, Baton Rouge, LA (US)

(72) Inventors: Adam T. Melvin, Zachary, LA (US); Elizabeth Martin, Baton Rouge, LA (US); Athena Lindsay, Baton Rouge, LA (US); Conner Allison, Baton Rouge, LA (US); Daniel Augustin, Baton Rouge, LA (US); Angelle Leger, Baton Rouge, LA (US); Erika Lewis, Baton Rouge, LA (US); Emmaline Miller, Baton Rouge, LA (US)

(21) Appl. No.: 17/223,630

(22) Filed: Apr. 6, 2021

Related U.S. Application Data
(60) Provisional application No. 63/005,836, filed on Apr. 6, 2020.

(51) Int. Cl. C12M 1/22; C12N 5/00

(52) U.S. Cl. CPC C12M 1/22; C12N 5/0062

(57) ABSTRACT

Provided are cell plate inserts that include an interior lip and legs on a bottom side. The cell plate inserts can have a diameter that provides a snug fit within a cell plate or well. The cell plate inserts can include a cell support membrane disposed on an interior lip of the cell plate insert. The cell plate inserts can be included in systems for cell co-culture, where cells cultured in a top chamber formed by the cell plate insert do not infiltrate cells cultured in a bottom chamber formed by the cell plate insert.

### CROSS-REFERENCE TO RELATED APPLICATIONS

This application claims the benefit of and priority to U.S. Provisional Application Ser. No. 63/005,836, having the title “SYSTEMS FOR CELL PLATE CO-CULTURE”, filed on Apr. 6, 2020, the disclosure of which is incorporated herein by reference in its entirety.

### BACKGROUND

Cellular communication has been studied extensively by scientists, clinicians and engineers, as it is a critical component in development and disease. In order to communicate, cells emit extracellular signaling molecules (e.g., cytokines and growth factors), which are recognized by surface receptors on nearby cells. This communication regulates if a cell will proliferate (increase in number), migrate, or undergo apoptosis (cell death). Understanding cell-cell communication is especially critical in diseases such as cancer. Solid tumors are composed of multiple cell types (cancer cells, endothelial cells, immune cells, adipocytes, fibroblasts) that can contribute to cancer progression and resistance to therapy. It is known that cancer progression and resistance to therapeutic intervention can arise from communication between cancer cells and the surrounding non-cancer cell populations.

Further understanding of this relationship can lead to developing improved adjuvant breast cancer therapies. Traditional approaches to co-culture two different types of cells are done by direct cell contact using a permeable insert (such as a Transwell® assay). This technology involves plating cells above and below a semi-permeable membrane to allow for secreted factors to diffuse to each cell line. One constraint of this current method is that the technique does not yield enough cells for thorough cellular analysis such as PCR or Western Blot. Another difficulty is successfully separating cell lines after co-culture. It can also be challenging to overcome these problems while still achieving adequate cell line communication. These needs and other needs are satisfied by the present disclosure.

### SUMMARY

Embodiments of the present disclosure provide cell plate inserts, systems for cell co-culture, and the like.

An embodiment of the present disclosure includes cell plate inserts that include an interior lip and legs on a bottom side. The cell plate inserts can have a diameter that provides a snug fit within a cell plate or well.

The cell plate inserts can include a cell support membrane disposed on an interior lip of the cell plate insert.

An embodiment of the present disclosure includes systems for cell co-culture, including a cell plate insert as above.

Other compositions, apparatus, methods, features, and advantages will be or become apparent to one with skill in the art upon examination of the following drawings and detailed description. It is intended that all such additional compositions, apparatus, methods, features and advantages be included within this description, be within the scope of the present disclosure, and be protected by the accompanying claims.

### BRIEF DESCRIPTION OF THE DRAWINGS

Further aspects of the present disclosure will be more readily appreciated upon review of the detailed description of its various embodiments, described below, when taken in conjunction with the accompanying drawings. The components in the drawings are not necessarily to scale, emphasis instead being placed upon clearly illustrating the principles of the present disclosure.

FIG. 1 is a diagram of a Transwell® insert, which is an existing qualitative tool used to study cell migration and perform cellular co-culture that consists of a lower compartment containing one cell line and a top compartment containing another cell line. Soluble chemicals will diffuse through the polycarbonate membrane separating the two sections.

FIG. 2 provides a comparison of RNA extracted between different co-culture platforms.

FIGS. 3A-3B are examples of a prototype in accordance with embodiments of the present disclosure. FIG. 3A shows an exploded view of the 3D-printed plate insert incorporated into a 10 cm tissue culture dish. The insert houses an agarose slab, which replaces the polycarbonate membrane. Two different cell types are cultured on the bottom of the dish and on the top of the agarose to mimic the diffusion of the Transwell insert while allowing for directly visualization and facile harvesting of cells for post-experimental analysis.

FIG. 3B is a camera image of a 2 mm Agarose layer fabricated and taken off from the base mold, which was supported by the 3D insert. FIGS. 3C-3D provide additional examples of possible design and schematic of the insert. FIG. 3C shows a top view. FIG. 3D provides a CAD model of the insert and an exploded view of the insert incorporated into a 10 cm tissue culture dish. Two different cell types can be cultured on the bottom of the dish and on the top of the agarose allowing diffusion of biomolecules while directly imaging both layer and facile harvesting of cells for post experimental analysis.

FIGS. 4A-4C are examples a prototype of the insert in accordance with embodiments of the present disclosure. FIG. 4A is a top view, FIG. 4B is a side view with dimensions of the insert, and FIG. 4C is an example of a CAD model of the insert.

FIGS. 5A-5B provide experimental validation of mass transfer and computational simulation of oxygen transfer across the agarose slab in accordance with embodiments of the present disclosure. FIG. 5A shows experimental measurement of the mass transfer of 500 kDa FITC-Dextran from the bottom chamber to the top chamber. FIG. 5B provides COMSOL simulations showing oxygen transfer from the top chamber to the bottom chamber reaching a steady state within 24 hours.

FIGS. 6A-6C provide images of MDA-MB-231 cell growth and morphology in a plate insert according to embodiments of the present disclosure. FIG. 6A shows single tissue culture plastic (Control-TCP). FIG. 6B shows agarose coated in a TCP. FIG. 6C shows cells cultured in the plate insert bottom (TCP) and top (agarose). All images were collected every 24 h during experimentation. Data included here are representative images of the entire plate.

FIGS. 7A-7C demonstrate ASC cell growth and morphology in a plate insert according to embodiments of the present disclosure. FIG. 7A shows single tissue culture plastic (Control-TCP). FIG. 7B shows agarose coated in a TCP. FIG. 7C shows cells cultured in the plate insert bottom (TCP) and top (agarose). All images were collected every 24 h during experimentation. Data included here are representative images of the entire plate.

FIGS. 8A-8C demonstrate HeLa cell growth and morphology in a plate insert according to embodiments of the present disclosure. FIG. 8A shows single tissue culture plastic (Control-TCP). FIG. 8B shows agarose coated in a TCP. C) Cells cultured in the plate insert bottom (TCP) and top (agarose). All images were collected every 24 h during experimentation. Data included here are representative images of the entire plate.

FIG. 9 demonstrates terminal viability staining the MDA-MB-231 cells cultured for 72 hours in the plate insert in accordance with embodiments of the present disclosure.

The drawings illustrate only example embodiments and are therefore not to be considered limiting of the scope described herein, as other equally effective embodiments are within the scope and spirit of this disclosure. The elements and features shown in the drawings are not necessarily drawn to scale, emphasis instead being placed upon clearly illustrating the principles of the embodiments. Additionally, certain dimensions may be exaggerated to help visually convey certain principles.

FIGS. 10A-10B shows MDA-MB-231 cell growth in TCP, agarose, and 3D plate inserts via alamar blue assay insert in accordance with embodiments of the present disclosure. The smaller version of 3-cm insert in 6-well plate was used for growth study using MDA-MB-231 cells where a 6-well plate was used as tissue culture plastic (TCP) for single culture as control and TCP with agarose layer for single culture. The cell density was 50,000 cells for each well or insert with 3 mL appropriate media in the bottom of the well and 2 mL media for the top of the insert plate. All experiments were conducted in triplicates using Alamar blue assay at four time-points of 24, 48, 72, and 92 hours.

FIG. 11 shows cell growth and morphology in the 3D cell plate insert in accordance with embodiments of the present disclosure. MDA-MB-231 were grown on top of insert-agarose and ASCs on the bottom of insert TCP. All images were collected every 24 hours during experimentation. Data included here are representative images of the entire plate.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the apparatus and methods disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

As used herein, the following terms have the meanings ascribed to them unless specified otherwise. In this disclosure, “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

### Definitions

Agarose, as used herein, is a polysaccharide derived from agar.

### General Discussion

In accordance with the purpose(s) of the present disclosure, as embodied and broadly described herein, embodiments of the present disclosure, in some aspects, relate to cell plate inserts and systems for cell co-culture.

The present disclosure provides for a cell plate insert including an interior lip (e.g. a support shelf) and legs on a bottom side. The cell plate insert has a diameter to provide a snug fit within a cell plate. Advantageously, the insert can be easily incorporated into standard tissue culture dishes. In some embodiments, the cell plate insert is optically transparent such that the contents of the plate or well can be viewed. In some embodiments, the cell plate insert can be 3D-printed. The cell plate insert can be made from any appropriate biocompatible material, from 3D printing thermoplastics (e.g., Polylactic Acid (PLA), Acrylonitrile Butadiene Styrene (ABS), PolyAmide (PA), High Impact Polystyrene (HIPS), and Thermoplastic elastomer (TPE)), or other materials used in cell culture vessels (e.g., polystyrene, some polycarbonate, stiff resins, glass), or other materials as can be envisioned by one of ordinary skill in the art.

The cell plate insert described herein can offer several advantages over existing technology including: (i) direct visualization of cells during experimentation, (ii) physical separation of the two cell lines to prevent infiltration from the top chamber to the bottom chamber, and (iii) dramatically increased yields of cells, protein, and DNA/RNA for post-experimental analysis.

Embodiments of the present disclosure include a cell plate insert as above having a cell support membrane disposed on the interior lip of the cell plate insert. In various embodiments, the cell support membrane includes agarose, or is made of agarose.

The present disclosure also provides for a system for cell co-culture that includes a cell plate insert, an interior lip on a top side and legs on a bottom side. The cell plate insert can have a diameter to provide a snug fit within a cell plate. In various embodiments, the system can include a cell support membrane that can be disposed on the interior lip of the cell plate insert. The plate insert can have an interior lip to support a cell culture support media. The lip can be on a top side, a bottom side, or both. When the cell support membrane is disposed on the cell plate insert, the cell plate insert forms a top chamber and a bottom chamber, wherein a first cell line can be cultured in the top chamber, and a second cell line can be cultured in the bottom chamber. The cell lines can be the same or different.

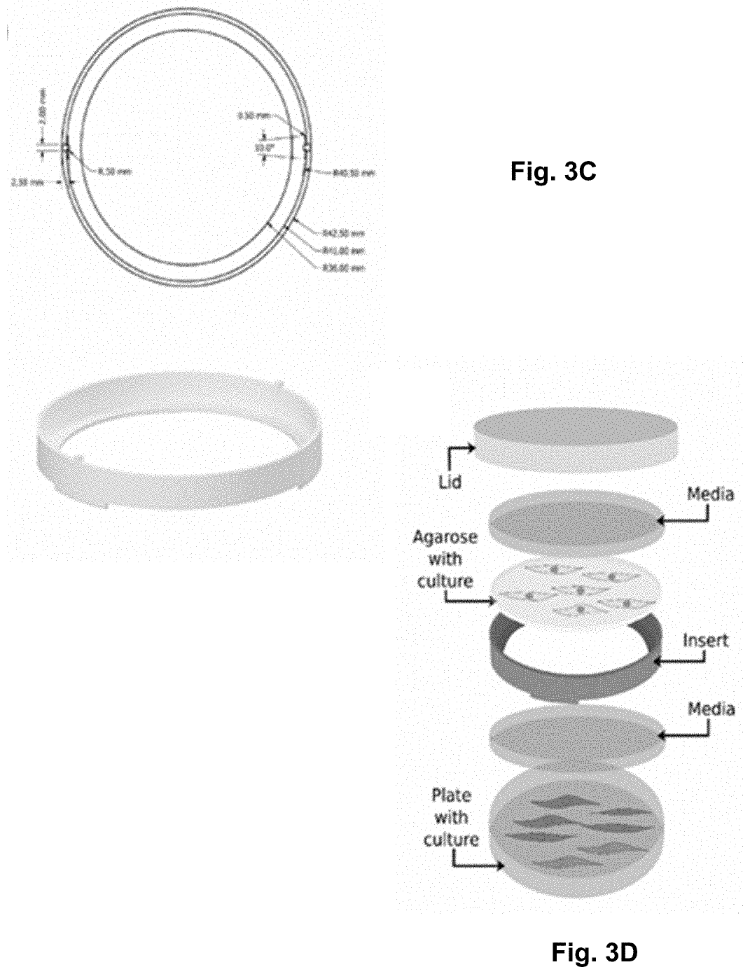

The cell culture support media can be an inert, non-cytotoxic matrix forming a membrane (e.g. an agarose membrane, alginate membrane, collagen membrane, hyaluronic acid membrane) that allows for the physical separation of at least two cell lines while still allowing for the chemical communication between the two via length-scale diffusion (FIGS. 3A-3B). In some embodiments, a third cell line could be entrained in the membrane, such as a collagen or alginate membrane.

In various embodiments, the cell plate includes a first tissue culture substrate. The cell plate insert can be inserted on top of the tissue culture substrate. In various embodiments, the first tissue culture substrate includes tissue culture plastic (TCP). In some embodiments, the tissue culture substrate can include glass.

In various embodiments, the cell plate insert includes legs that are of a height sufficient to provide an equal distance between the first tissue culture substrate and the cell culture support media. The cell plate insert can have a diameter to provide a snug fit within a cell plate. The snug fit ensures physical separation of the two cell lines to prevent infiltration from the top chamber to the bottom chamber. The number of legs can vary between 3 to about 10 legs, or 4 legs. In some embodiments, the legs can be replaced by a continuous bottom rim. Advantageously, the cell plate allows for oxygen permeability between chambers while preventing cell infiltration.

In a non-limiting embodiment, the cell plate insert can be manufactured to dimensions to accommodate a 10 cm diameter cell plate that is about 13 mm deep. The overall height can be about 10 mm high, including legs that can be about 1 mm to 4 mm, about 1 to 3 or about 2 mm high. The lip can be about 1 mm to 3 mm thick or about 2 mm thick. The lip can be placed about 3 mm below the upper edge of the cell plate insert. Such a configuration can provide a spacing between the first tissue culture substrate on the bottom of the plate and the cell culture support media, wherein the spacing is about 1 mm to about 6 mm, about to 2 mm to about 5 mm, or about 5 mm. The insert can have a diameter of about 95 mm to 99.9 mm, about 96 to about 99 mm or about 95 mm, and the walls of the insert can be about 2 mm to about 6 mm thick, about 2 mm to 5 mm thick, or about 3 mm thick. The lip can project from the interior wall of the cell plate insert sufficient to form support shelf for the cell support membrane. The lip can project about 0.5 to 4 mm, about 1 mm to 3 mm, or about 2 mm. As can be envisioned by one of ordinary skill in the art, these dimensions can be scaled or optimized for use in cell plates of varying sizes.

In another non-limiting embodiment, the cell plate insert can be manufactured to dimensions to accommodate a 36.4 diameter cell plate having a diameter of about 33 mm to 37 mm (e.g. a standard 6-well plate, such as a Corning) that is about 20 mm deep. The overall height can be about 10.15 mm high, excluding legs that can be about 1 mm to 4 mm, about 1 mm to 3 mm or about 2.8 mm high. In some embodiments, the legs can be about 8.8 mm wide The lip can be about 1 mm to 4 mm thick or about 3 mm thick. The lip can be placed about 3 mm below the upper edge of the cell plate insert. Such a configuration can provide a spacing between the first tissue culture substrate on the bottom of the plate and the cell culture support media, wherein the spacing is about 1 mm to about 6 mm, about to 2 mm to about 5 mm, or about 5 mm. The insert can have a diameter of about 28 mm to 35 mm, about 30 to about 34 mm or about 33 mm, and the walls of the insert can be about 1 mm to about 6 mm thick, about 2 mm to 5 mm thick, or about 2 mm thick. The lip can project from the interior wall of the cell plate insert sufficient to form support shelf for the cell support membrane. The lip can project about 0.5 to 4 mm, about 1 mm to 3 mm, or about 2 mm. As can be envisioned by one of ordinary skill in the art, these dimensions can be scaled or optimized for use in cell plates of varying sizes.

In other embodiments, as can be envisioned by one of ordinary skill in the art, the plate can be scaled to fit other sizes or shapes of petri dishes or well plates. Such plates can include but are not limited to small wells such as in 12 well plates or larger petri dishes such as a 15 cm diameter petri dish. In some embodiments, such as for a multi-well plate, the cell plate inserts can be connected such that multiple cell plates can be simultaneously inserted in a multi-well plate.

In various embodiments, the system can include a second tissue culture substrate disposed on top of the cell support membrane.

The system can also include a cell plate lid and/or a base for forming the cell support membrane.

To date, there are no other commercially-available options to perform cellular co-culture experiments beyond the Transwell insert, so there is a significant need for alternative platforms that allow for greater cellular yields for post-experimental analysis. The technology developed in the present disclosure possesses the capabilities to study cell-to-cell communication which can allow for a greater fundamental understanding of how cell-to-cell communication results in the increase or decrease of drug efficacy in clinical trials. The system described herein is user-friendly, cheaper, and allows for dynamic and tunable co-culture conditions while maintaining similar growth patterns as compared to off-chip controls in tissue culture plastic (TCP). Experimental and COMSOL simulations show the rapid diffusion of biomolecules and oxygen across the hydrogel, reaching a steady-state within ˜24 hours. The plate insert was used to study the interactions between three different breast cancer cell lines, triple-negative-MDA-MB-231, HeLa, and ER+(MCF7) co-cultured with adipose-derived stem cells (ASCs). Using this novel co-culture approach, enhanced cellular growth and overexpression of EMT-associated genes were identified in the MDA-MB-231 cell line when they were cultured in the presence of ASCs.

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Example 1

### BACKGROUND

Currently, neoadjuvant therapies have been identified that target non-cancer cells within the tumor. Examples of this include immunotherapies such as T-cells and PD-1, inhibition of endothelial cell vascular formation through blocking VEGF, or targeting fibroblasts to inhibit matrix production.³ While many adjuvant therapies have been proposed, the complexity of solid tumors and cancer cell-to-cell communication with the surrounding population makes pharmacological intervention complex. In a recent research study by Paino and colleagues, the impact of cancer cells on adipose-derived mesenchymal stem cells (ASCs) was studied by co-culturing ASCs with breast cancer cell line MCF7.⁴ After 21 days of co-culture study, ASCs triggered angiogenesis with highly vascularized tumor while they did not differentiate; the findings were very significant for the adipose tissue autografting in patients with cancer. Sabol et al. performed another research to examine the relationship between ASCs and MCF7 breast cancer cell line.⁵ They have demonstrated that the ASCs from obese patients stimulated tumor growth and metastasis in estrogen receptor breast cancer due to increased leptin secretion. Interaction of ASCs and breast cancer studied by Sakurai et al. observed similar results, and they found a calcium-binding protein upregulated six-fold in MCF7 cells when co-cultured with ASCs.⁶

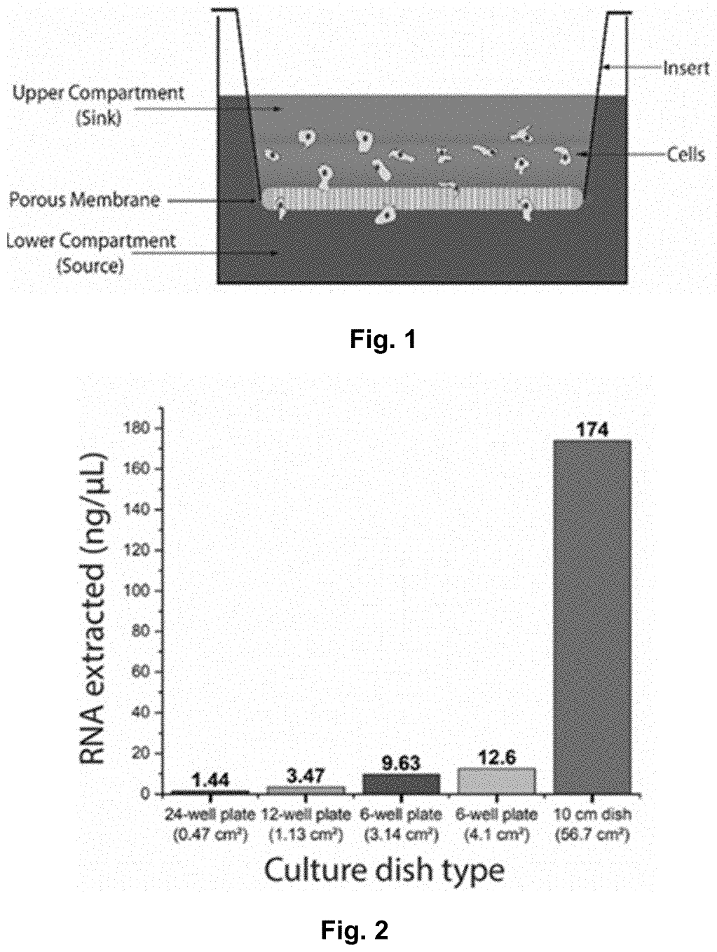

There is significant interest in gaining a more complete understanding of the intracellular signaling mechanisms driving biomolecule secretion and reception as recent studies have found that cells are phenotypically and genotypically different when cultured alone versus cultured alongside different cell types.^{7,8} To better understand this paradigm and identify novel neoadjuvant therapies, continual dynamic co-culture of ASCs and cancer cells is required. One approach to study cell-to-cell communication employs conditioned medium.⁹ This involves culturing one cell line in flaks followed by the collection and transfer of only the media to another cell line in a different flask. While this approach is incredibly easy to use, it is limited to one-way communication and lacks any dynamic interactions between the two cell lines. Therefore cell-to-cell communication is naïve and not representative of a correlative cellular response. Advances on this method include the industry standard for cell-to-cell interactions: co-culture, a process in which more than one cell line is grown in the cell culture dish. The most commonly used technique is the Transwell insert (or Boyden Chamber, Transwell plate).¹⁰ This approach utilizes inserts that fit into 24-, 48-, or 96-well plates that contains a polycarbonate membrane to separate two cell lines (FIG. 1). The Transwell insert was primarily designed to study cell migration. It therefore suffers from several weaknesses as a tool for dynamic cellular co-culture including (i) an inability to directly visualize the cells during an experiment, (ii) the invasion of the cells in the top chamber into the polycarbonate membrane and the bottom chamber preventing the collection and continual propagation of cells for long term studies, and (iii) very low cellular yields for post-experimental analysis.^{7,11}

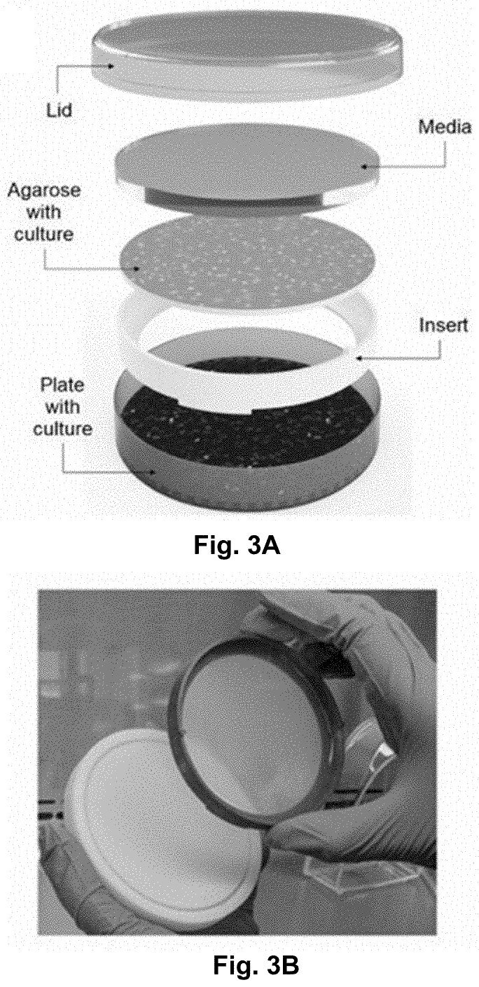

Moreover, Transwell inserts are limited by their ability to collect sufficient cell number for PCR and western blot analysis, two standards in the validation of therapeutic efficacy. Typical cell seeding protocols for Western blot and PCR analysis require cell numbers up to 1-2 million cells per experiment.¹² Due to cell density restriction, these numbers are not achievable on the smaller Transwell inserts. Average cell seeding densities on 24-, 48-, and 96-well inserts at maximum cell confluence for standard epithelial and mesenchymal cell lines are 240,000 cells, 120,000 cells, and 40,000 cells respectively.¹³ This is made further evident by the comparison of obtainable RNA from different cell culture wells and dishes of different sizes (FIG. 2), where standard qRT-PCR reaction kits require 100 ng/ul per reaction. To obtain the RNA extraction data, RNA was extracted from a 10 cm dish (control, top+bottom of insert). The RNA extraction numbers were extracted from control+top+bottom, the RNA extracted per cm² of culture area was calculated. The obtained value was scaled to the respective culture dishes based on surface area.

Recently, microfluidic devices have attempted to address this need by providing an alternative platform for simultaneous co-culture. Byrne et al. developed a microfluidic device based on polydimethylsiloxane (PDMS)-glass to study cell-to-cell signaling using mouse macrophage cells and human kidney cells where diffusion of media occurred across narrow channels that connected the microcompartments.¹⁴ The results demonstrated that TNF-α secreted by macrophage stimulated the kidney cells. Ma et al. used a similar microfluidic device consisting of multiple culture chambers interconnected with microchannels to study cell-to-cell communication between fibroblasts and tumor cells.¹⁵ Cheng et al. developed an agarose-based microfluidic approach for the study of the chemotactic invasion of HL-60 (neutrophil-like) cells in 2D and dendritic cells in 3D collagen gel.^{18,17} The major pitfalls of those microfluidic devices encountered shearing formation by the media flow. There are some other groups who developed the co-culture approaches by modifying the existing methods. Thomsen et al. developed an agarose hydrogel-based microwell co-culture platform where breast cancer cells (MDA-MB-231) co-cultured successfully with mesenchymal stromal cells for several weeks.¹⁸ They demonstrated the enhanced proliferation in cancer cells, which verifies the exchange of secreted biomolecules between both types of cells through agarose hydrogel while cells remain in the agarose microwell. Agarose also has been used in another co-culture approach described by Young and colleagues in which they seeded cells on a cellulose scaffold and separated distinct cells by agarose layer in a stacked-layer culture model to study the influence of cancer-associated fibroblasts (CAFs) on cancer cells.¹⁹ The results indicate that the squamous carcinoma cells increased proliferation and invasiveness due to the stimulation by the CAFs in the new co-culture model. While informative, microfluidic devices suffer from their own limitation. They require external equipment (e.g. pumps) and still suffer from very low RNA yields (less than 1.44 ng/μL).²⁰ Thus, there is a need for a new type of continual co-culture platform that is easy-to-use and allows for very high post-experimental yields of cells, proteins, and DNA/RNA.

### 3D-Printed Plate Insert Fabrication

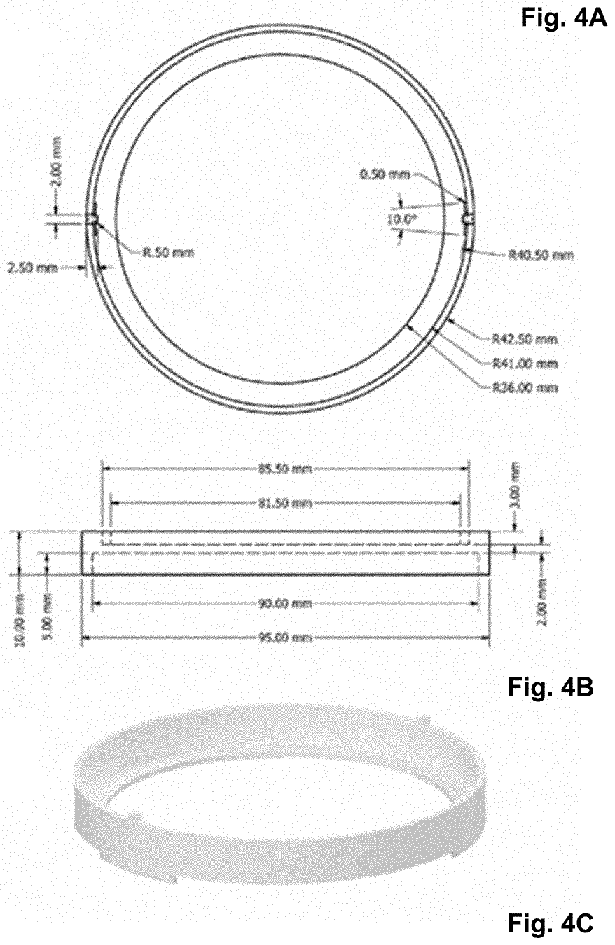

The plate insert was designed to create a mass transfer system vertically separated by an agarose membrane in a petri dish. The plate insert is easy to manufacture using a 3D printer, which has led to several design improvements to produce an optimal design (FIGS. 3A-3B). All designs were modeled using AutoDesk Inventor and printed in the LSU Chevron Center for Engineering Education using a biocompatible, non-toxic, 3D printing resin. The plate insert was designed to fit into a 10 cm tissue culture dish to allow for the maximal number of harvested cells for post-experimental analysis (FIG. 4A). A base mold was also fabricated to allow for the facile generation of the agarose slab inside of the plate insert (FIG. 4B). This allowed for easy generation of the agarose membrane while simultaneously ensuring a uniform fit of the agarose to the insert to avoid any infiltration of the cells from the top layer to the bottom layer. The design features include three 2 mm tall legs and the 3 mm lip of the Insert below the agarose layer, providing a cumulative 5 mm spacing which keeps the consistent spacing between the bottom cell culture and agarose barrier (FIG. 4C). The dimensions of the top and side view of the Insert are shown in FIGS. 4A and 4B. The plate insert was designed to hold adequate media on the top and bottom layers and has an increased wall height for use with deeper culturing dishes (>13 mm). FIG. 3A shows an exploded view of the whole system where the agarose is separated from the insert but, in reality, it would be seated on the support shelf of the insert.

### Device Characterization

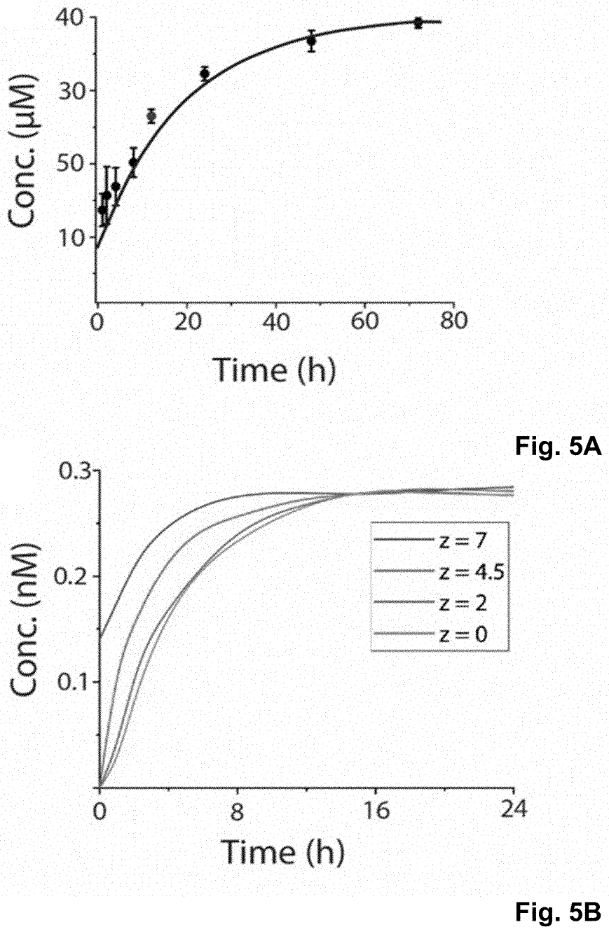

The mass transfer in the system was first characterized both experimentally and computationally before the plate insert could be utilized for experimentation. Time-dependent mass transfer diffusion studies of oxygen and biomolecules (representing paracrine signals like growth factor or cytokines) were simulated across the agarose barrier to ensure adequate supply for cell growth. A passive diffusion experiment monitoring the mass transfer of 500 kDa FITC(fluoroisothiocynate)-dextran was conducted over a 72 h period, approximately the same duration as culturing experiments. FITC-Dextran molecule was selected for diffusion testing for several reasons: availability/quantity of research on the molecule, similar size to large secreted biomolecules, and ease of quantification. To begin testing, 16 mL of 20 μM FITC-dextran solution was pipetted to the bottom of a petri dish while 16 mL DI water was pipetted on top of the agarose of the Insert. Over the 72 h experiment, samples were drawn at each time point (0, 1, 2, 4, 8, 12, 24, 48, 72 h) and measured absorbance at 488 nm in a NanoDrop® spectrophotometer. The analyzed data (FIG. 5A) show that the FITC-Dextran diffusion reached a steady state within ˜24 h. These results confirm that the agarose on the plate insert allowed for indirect communication between two different cell types cultured on two layers to facilitate co-culture studies of breast cancer cells and ASCs. COMSOL simulations of the mass transfer were also preformed to validate the experimental studies (data not shown). In the simulation, the water layers used the ‘Diffusion of Dilute Species model’ while the diffusion through the agarose layer used ‘Diffusion through Porous Media model’ in COMSOL. The porosity of the agarose was approximated as 0.971. The simulation used the physical properties of water and fluoroisothiocynate (FITC)-Dextran at 40 KDa to mimic biomolecular diffusion through the agarose membrane. The simulation demonstrated that cell cultured on both the top agarose layer and the bottom tissue culture plastic (TCP) reached a steady-state after 36 h. To confirm the proper diffusion of oxygen across the device, additional COMSOL simulations were performed. For this simulation, the static ambient air was modeled to diffuse through the agarose membrane (FIG. 5B). In the figure, the z value of 7 indicates that the diffusion begins from the agarose membrane at 7 mm height from the bottom of the dish atz=0. An approximate steady-state was observed at ˜24 h confirming that the cells in the bottom layer will not be exposed to hypoxic conditions.

### Agarose Membrane Preparation

The 3% (w/v) agarose solution in extracellular buffer (ECB: 5.036 mM HEPES, 136.89 mM NaCl, 2.68 mM KCl, 2.066 mM MgCl₂.6H₂O, 1.8 mM CaCl₂.2H₂O, and 5.55 mM glucose) was used to form the agarose membrane into the insert. Before pouring the autoclaved agarose, the base feet of the plate insert were inserted into the circular channel of the mold in the biosafety hood. Then 15 mL of the heated, sterile, liquid agarose solution is pipetted over the mold to create a 2 mm thick membrane. The agarose was allowed to solidify into a circular slab for 30 min. The base mold allowed for a tight seal between the agarose and the insert. This seal is important for both the structural integrity of the agarose and the inhibition of cellular migration. The agarose was next pre-treated with 0.1 mg/mL poly-D-lysine in 1× phosphate-buffered saline (PBS: 137 mM NaCl, 10 mM Na2HPO4, 27 mM KCl, and 1.75 mM KH2PO4 at pH 7.4) to induce cationic charges that facilitate the attachment of the anionically charged cells to the agarose surface. 3 mL of poly-D-lysine solution was incubated on the agarose for 2 hours at room temperature in the biosafety hood. The poly-D-lysine solution was aspirated followed by 5X washing with serum-free culture media. The treated agarose was left in 4° C. overnight before the experiment begins. Single culture experimentation using the 3D-printed plate insert

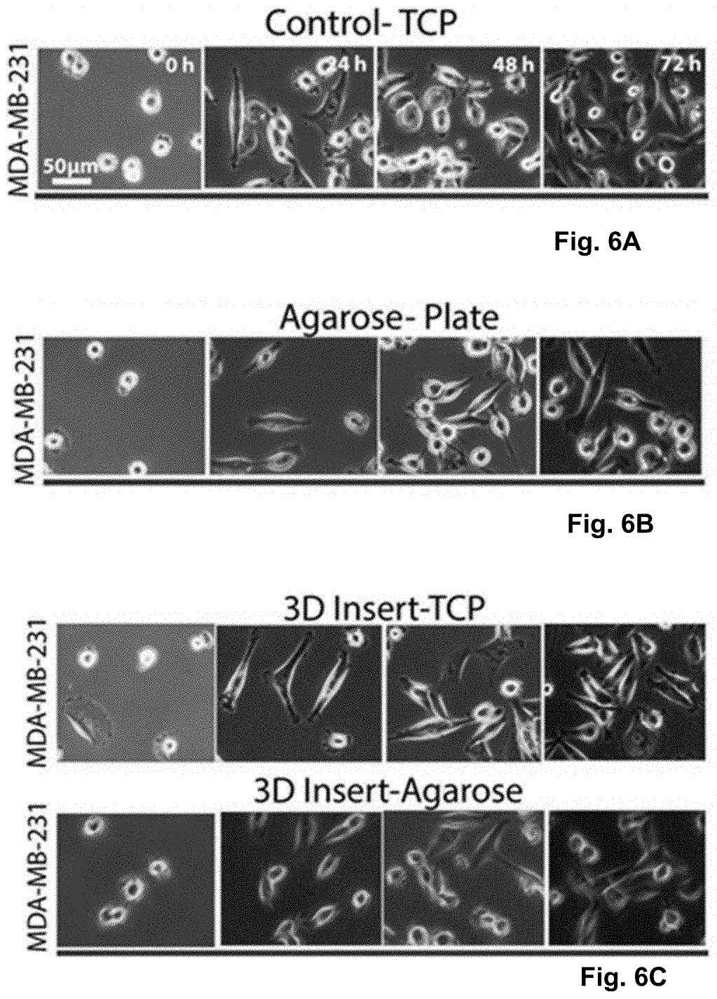

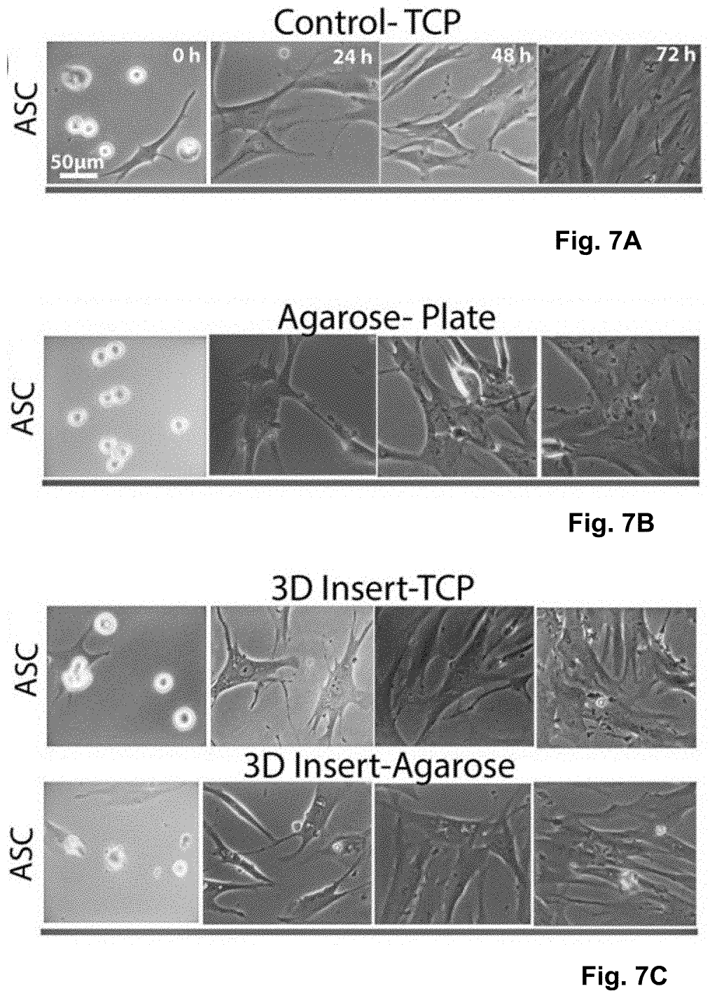

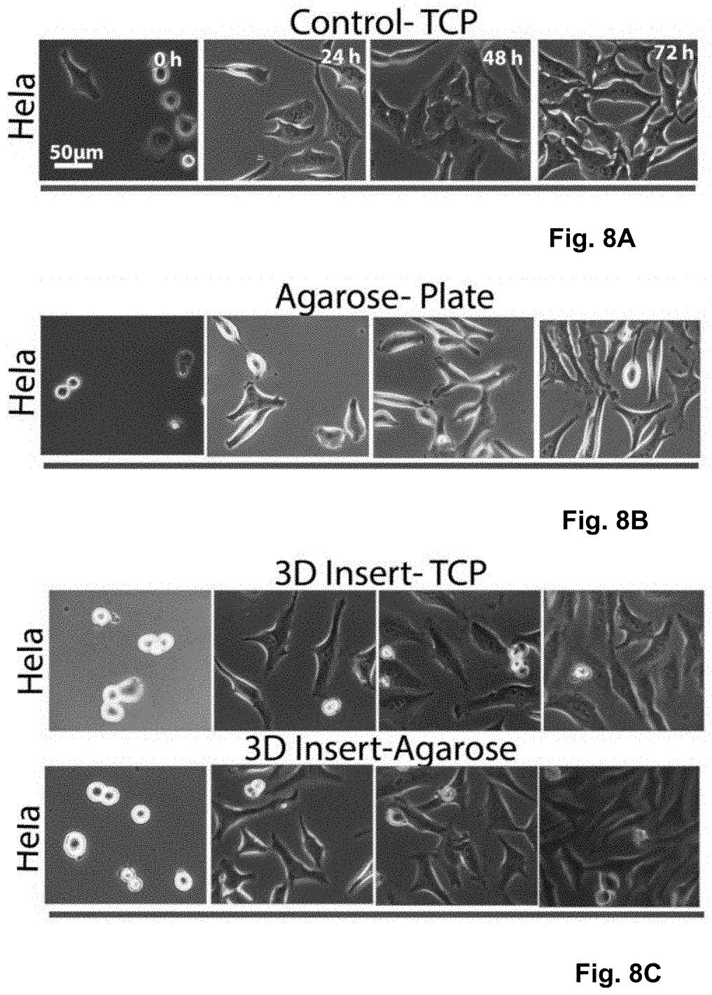

The poly-D-lysine treated agarose with the plate insert was washed with the cell culture media (DMEM supplemented with 10% v/v HyClone Cosmic Calf Serum, 1% MEM Essential Amino Acids, 1% MEM Non-Essential Amino Acids, 1 mM Sodium Pyruvate, and 6 μL insulin/500 mL media) five times before the cell seeding. Next, one of the two cell lines was seeded with appropriate density on the bottom of the 10 cm culture plate with additional 15 mL of culture media. The plate insert with treated agarose membrane was placed gently into the 10 cm dish, without trapping air bubbles underneath. The second cell line was seeded on the treated agarose followed by the addition 15 mL of cell culture media. Finally, the culture plate lid was placed on top of the dish to maintain sterility. Control single culture experiments were first performed to confirm that cancer cell lines and adipose-derived stem cells (ASCs) behaved the same in the plate insert as they did on traditional tissue culture plastic (TCP). Each cell line was also cultured on separate Petri dishes with or without agarose to evaluate any morphological changes in the novel plate insert co-culture system. The petri dish with agarose was prepared by pipetting 15 mL of the same agarose in a petri dish (TCP) followed by similar poly-D-lysine treatment. All cultures were incubated for 72 h in a humidified incubator (VWR) at 37° C. with 5% carbon dioxide (CO2). Images were collected every 24 h by using a Zeiss Primo Vert phase-contrast microscope equipped with an Axiocam105 color digital camera. All images were further processed in ImageJ (NIH) software and Adobe Illustrator. No change was observed in the growth rate or morphology of triple negative breast cancer cells (MDA-MB-231, FIGS. 6A-6C), ASCs (FIGS. 7A-7C), or ovarian cancer cells (HeLa, FIG. 8A-8C) cultured on tissue culture plastic (Control-TCP), agarose on TCP (Agarose-Plate), and in the plate insert in which the bottom is TCP (3D Insert-TCP) and the top is agarose (3D Insert-Agarose). These essential control experiments confirm that the plate insert can facilitate the culture of cancer and stromal cell lines with a minimal effect on cellular behavior. This is important because any changes observed in cellular behavior (e.g., growth, morphology, genotype) is due to the presence of a second cell type and not the technology.

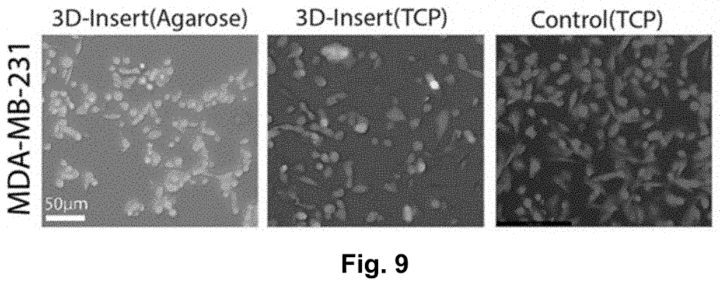

Additionally, terminal viability staining was performed on the MDA-MB-231 cells cultured for 72 hours in the plate insert (FIG. 9). Cell viability was assessed by Calcein AM (live, green) and Ethidium homodimer 1 (dead, red) stains using fluorescence microscopy. Cellular fluorescence was visualized using a Leica DMi8 inverted microscope outfitted with a FITC filter cube, 20× objective (Leica HC PL FL L, 0.4× correction), and phase contrast and brightfield applications. Digital images were acquired using the Flash 4.0 high speed camera (Hamamatsu) with a fixed exposure time of 500 ms for FITC, DAPI, and Rhodamine filters, and 10 ms for brightfield. Image acquisition was controlled using the Leica Application Suite software. The cells cultured on the bottom TCP layer and top agarose layer exhibited a similar degree of viability which was comparable to control cells cultured on TCP (FIG. 9). This confirmed that the plate insert did not alter cell viability and that any future changes in viability during dynamic co-culture will be due to the presence of a second cell line and not the plate insert technology.

A similar approach to what was described above has been performed under dynamic co-culture conditions. For these initial experiments, MDA-MB-231 cells were cultured on the top agarose layer and ASCs were cultured on the bottom TCP layer. After 72 h of co-culture, both cell lines were collected followed by the successful extraction of cellular DNA and RNA with impressive yields (˜100-400 ng/μL) and purity (A260/A280 values at 2.0) sufficient for both PCR and Western blot analysis. This analysis is currently underway to identify changes in gene expression in both MDA-MB-231 cells and ASCs under dynamic co-culture conditions compared to single culture experiments. Future studies will follow a similar approach; however, they will investigate increased drug resistance in MCF7 cells co-cultured with ASCs.

### CONCLUSIONS

The 3D-printed plate insert co-culture system described herein possesses the potential to replace the existing Transwell assay for co-culture studies. The system facilitates dynamic co-culture between two cell types without interrupting the viability and morphology of the cells. The plate insert allows bright field imaging during the experiment and delivers a sufficient amount of cells for further analysis, which features make the plate insert unique. Initial counts of collected cells indicate suitable numbers for later gene expression analysis through methods such as PCR and Western Blotting. The COMSOL simulations and experimental FITC-dextran tests confirm sufficient mass transfer across the device and between the two different cell types validating that the desired cellular biomolecules can diffuse through the agarose-insert interface. This system also does not result in the infiltration of cells from the top layer to the bottom (a significant limitation of the Transwell assay) preventing the cross-mixing of cells. This makes the plate insert approach an ideal choice for drug screening and discovery.

Additional information can be found in Appendices A and B.

### REFERENCES

(1) Korkaya, H.; Liu, S.; Wicha, M. S. Breast Cancer Stem Cells, Cytokine Networks, and the Tumor Microenvironment. Journal of Clinical Investigation. 2011. https://doi.org/10.1172/JCI57099.
(2) Guo, S.; Deng, C. X. Effect of Stromal Cells in Tumor Microenvironment on Metastasis Initiation. International Journal of Biological Sciences. 2018. https://doi.org/10.7150/ijbs.25720.
(3) Dykes, S. S.; Hughes, V. S.; Wiggins, J. M.; Fasanya, H. O.; Tanaka, M.; Siemann, D. Stromal Cells in Breast Cancer as a Potential Therapeutic Target. Oncotarget. 2018. https://doi.org/10.18632/oncotarget.25245.
(4) Paino, F.; La Noce, M.; Di Nucci, D.; Nicoletti, G. F.; Salzillo, R.; De Rosa, A.; Ferraro, G. A.; Papaccio, G.; Desiderio, V.; Tirino, V. Human Adipose Stem Cell Differentiation Is HighlyAffected by Cancer Cells Both in Vitro and in Vivo: Implication for Autologous Fat Grafting. Cell Death Dis. 2017, 8 (1), e2568. https://doi.org/10.1038/cddis.2016.308.
(5) Sabol, R. A.; Beighley, A.; Giacomelli, P.; Wise, R. M.; Harrison, M. A. A.; O'donnnell, B. A.; Sullivan, B. N.; Lampenfeld, J. D.; Matossian, M. D.; Bratton, M. R.; et al. Obesity-Altered Adipose Stem Cells Promote ER+ Breast Cancer Metastasis through Estrogen Independent Pathways. Int. J. Mol. Sci. 2019, 20 (6), 1-13. https://doi.org/10.3390/ijms20061419.
(6) Sakurai, M.; Miki, Y.; Takagi, K.; Suzuki, T.; Ishida, T.; Ohuchi, N.; Sasano, H. Interaction with Adipocyte Stromal Cells Induces Breast Cancer Malignancy via S100A7 Upregulation in Breast Cancer Microenvironment. Breast Cancer Res. 2017, 19 (1), 1-12. https://doi.org/10.1186/s13058-017-0863-0.
(7) Alvarez-Garcia, Y. R.; Ramos-Cruz, K. P.; Agostini-Infanzón, R. J.; Stallcop, L. E.; Beebe, D. J.; Warrick, J. W.; Domenech, M. Open Multi-Culture Platform for Simple and Flexible Study of Multi-Cell Type Interactions. Lab Chip 2018. https://doi.org/10.1039/c8lc00560e.
(8) Pietras, K.; Ostman, A. Hallmarks of Cancer: Interactions with the Tumor Stroma. Exp. Cell Res. 2010, 316 (8), 1324-1331. https://doi.org/10.1016/j.yexcr.2010.02.045.
(9) Strong, A. L.; Pei, D. T.; Hurst, C. G.; Gimble, J. M.; Burow, M. E.; Bunnell, B. A. Obesity Enhances the Conversion of Adipose-Derived Stromal/Stem Cells into Carcinoma-Associated Fibroblast Leading to Cancer Cell Proliferation and Progression to an Invasive Phenotype. Stem Cells Int. 2017, 2017. https://doi.org/10.1155/2017/9216502.
(10) Regier, M. C.; Alarid, E. T.; Beebe, D. J. Progress towards Understanding Heterotypic Interactions in Multi-Culture Models of Breast Cancer. Integr. Biol. (United Kingdom) 2016, 8 (6), 684-692. https://doi.org/10.1039/c6ib00001k.
(11) Warrick, J. W.; Murphy, W. L.; Beebe, D. J. Screening the Cellular Microenvironment: A Role for Microfluidics. IEEE Rev. Biomed. Eng. 2008. https://doi.org/10.1109/RBME.2008.2008241.
(12) Carriere, P. P.; Llopis, S. D.; Naiki, A. C.; Nguyen, G.; Phan, T.; Nguyen, M. M.; Preyan, L. C.; Yearby, L.; Pratt, J.; Burks, H.; et al. Glyceollin I Reverses Epithelial to Mesenchymal Transition in Letrozole Resistant Breast Cancer through ZEB1. Int. J. Environ. Res. Public Health 2015. https://doi.org/10.3390/ijerph13010010.
(13) Thermofisher Scientific. Useful Numbers for Cell Culture. Invitrogen 2005, 1. (accessed 5/14/19 https://www.thermofisher.com/us/en/home/references/gi bco-cell-culture-basics/cell-culture-protocols/cell-culture-useful-numbers.html)
(14) Byrne, M. B.; Trump, L.; Desai, A. V.; Schook, L. B.; Gaskins, H. R.; Kenis, P. J. A. Microfluidic Platform for the Study of Intercellular Communication via Soluble Factor-Cell and Cell-Cell Paracrine Signaling. Biomicrofluidics 2014, 8 (4), 1-9. https://doi.org/10.1063/1.4887098.
(15) Ma, H.; Liu, T.; Qin, J.; Lin, B. Characterization of the Interaction between Fibroblasts and Tumor Cells on a Microfluidic Co-Culture Device. Electrophoresis 2010. https://doi.org/10.1002/elps.200900776. (16) Cheng, S.; Heilman, S.; Wasserman, M.; Archer, S.; Shuler, L.; Wu, M. A Hydrogel-Based Microfluidic Device for the Studies of Directed Cell Migration {. 2007, 763-769. https://doi.org/10.1039/b618463d.
(17) Haessler, U.; Kalinin, Y.; Swartz, M. A.; Wu, M. An Agarose-Based Microfluidic Platform with a Gradient Buffer for 3D Chemotaxis Studies. Biomed. Microdevices 2009, 11 (4), 827-835. https://doi.org/10.1007/s10544-009-9299-3.
(18) Thomsen, A. R.; Aldrian, C.; Bronsert, P.; Thomann, Y.; Nanko, N.; Melin, N.; Rucker, G.; Follo, M.; Grosu, A. L.; Niedermann, G.; et al. A Deep Conical Agarose Microwell Array for Adhesion Independent Three-Dimensional Cell Culture and Dynamic Volume Measurement. Lab Chip 2018, 18 (1), 179-189. https://doi.org/10.1039/C7LC00832E.
(19) Young, M.; Rodenhizer, D.; Dean, T.; Arcangelo, E. D.; Xu, B.; Ailles, L.; Mcguigan, A. P. Biomaterials A TRACER 3D Co-CUlture Tumour Model for Head and Neck Cancer. 2018, 164.
(20) Sackmann, E. K.; Fulton, A. L.; Beebe, D. J. The Present and Future Role of Microfluidics in Biomedical Research. Nature. 2014. https://doi.org/10.1038/nature13118.

### Example 2

As indicated above, the majority of microfluidic devices suffer from a few limitations, including (i) an inability to collect/harvest the cells off-chip for post-experimental analysis, (ii) low cell yields, (iii) a require for on-chip interrogation using immunostaining, and (iv) the use of expensive external pumps to infuse the device with media. As such, while microfluidic devices are a powerful tool to perform dynamic co-culture applications, there are certain instances in which a different approach is needed. To address this limitation, a 3D-printed plate insert that incorporates an agarose hydrogel layer to serve as a semi-permeable membrane to create two distinct culture chambers was developed. These two culture chambers, above and below the agarose slab, are physically separated yet chemically connected (FIGS. 3C-3D). This allowed for the facile transport of biomolecules without the physical infiltration of cells from the top chamber to the bottom chamber that plagues the Transwell assay. Another advantage of the plate insert is that it is compatible with light microscopy allowing for the continuous observation of cells in both the top and bottom chambers. Two different-sized inserts were fabricated to fit into either a 10 cm petri dish or a 3 cm 6-well plate. The 10 cm insert was used for harvesting a maximum number of cells for PCR analysis (see Example 1, above), while the 3 cm insert was developed to perform cell growth studies that required a plate reader and established viability stains. For both inserts, the agarose hydrogel was coated with poly-D-lysine to facilitate cellular attachment, spreading, and growth of adherent cell lines in the top layer. The design approach is user-friendly, inexpensive, and allows for dynamic and tunable co-culture conditions while preserving similar growth trends as compared to commonly used tissue culture plastic (TCP). Mass transfer between the two chambers was evaluated both experimentally and via COMSOL simulations to confirm the rapid diffusion of biomolecules and oxygen across the hydrogel approaching a steady state within 18 hours and 6 hours, respectively. The 3D-printed plate insert approach allowed for the direct visualization of cell lines in both the top and bottom chambers to track cell growth and morphology. A proof-of-concept study using the plate insert was performed to investigate intercellular communication between breast cancer cells and adipose-derived stem cells (ASCs), which play a critical role in promoting metastasis and drug resistance in breast cancer patients. The co-culture approach allowed for the independent harvesting of both cell lines in numbers sufficient for downstream PCR analysis to identify changes in the genotypic and phenotypic signature of cancer cells.

A smaller version of the plate insert described in Example 1 was designed by scaling down the 10 cm diameter to 3 cm diameter to fit into the well of a 6-well plate (Corning, USA). Both inserts and bases were designed using Solidworks (MA, USA) and were fabricated using an Ender-3 Pro 3D printer using a 1.75 mm biocompatible, non-toxic, 3D printing filament (polylactic acid, PLA). The base mold's purpose was to assist in the pouring and solidification of the agarose slab prior to experimentation.

Computational Simulation of Mass Transfer in the Plate Insert Using COMSOL Multiphysics

COMSOL simulations were performed to model the mass transfer of biomolecules and oxygen between the two chambers above and below the plate insert's agarose membrane. The purpose of the model was to ensure that the soluble biomolecules excreted from the cells of one chamber were able to diffuse through the agarose membrane with media to the other chamber cells and validate that the ambient oxygen could reach the bottom chamber to ensure no hypoxic conditions. In the nutrient mass transfer simulation, the property of the media was assumed as water (density 995.6 kg-m⁻³, viscosity 7.97×10⁻⁴ Pa-s) and the diffusion through the media considered the ‘Diffusion of Dilute Species model’ while the diffusion through the agarose layer used ‘Diffusion through Porous Media model’. The porosity of the 3% (w/v) agarose was approximated as 0.971.¹³⁹ The simulation used the physical properties of 500 kDa fluorescein isothiocyanate (FITC)-dextran to mimic the upper threshold of potential biomolecules that could pass through the agarose membrane. The approximated initial concentration of the FITC-dextran in the bottom chamber was 10 μM. To confirm the proper diffusion of oxygen across the device, the COMSOL model used static ambient air to diffuse through the agarose membrane. The diffusion begins from the agarose membrane at 7 mm height from the bottom of the dish, which is denoted as the z value of 7 at the agarose surface and z=0 at the bottom of the Petri dish.

Experimental Characterization of Mass Transfer in the Plate Insert Using FITC-Dextran

The mass transfer in the system was validated experimentally using a passive diffusion experiment monitoring the mass transfer of 500 kDa FITC-dextran (Sigma, USA) from the bottom chamber to the top chamber over a 72 hours period. To begin testing, 15 mL of 40 μM FITC-dextran solution was pipetted to the bottom chamber of the dish, while 15 mL DI water was pipetted above the agarose in the insert. Samples were collected from the top chamber at nine different time points (0, 1, 2, 4, 8, 12, 24, 48, 72 hours) at five different positions. The samples were then measured for absorbance at 488 nm in a NanoDrop® spectrophotometer (Thermo Fisher Scientific, MA, USA).

Agarose Membrane Preparation

As described in Example 1 for the petri dish insert, the agarose membrane was formed by pouring liquid agarose into the insert in a biosafety hood. The 3% (w/v) agarose solution was prepared and autoclaved, as described in section 2.2.4. The base mold and plate insert were sterilized by immersing them in ethanol before assembly in the biosafety hood. Both the insert and base were left in the hood for ˜1 hour to dry. The insert was locked in a circular channel of the mold before pipetting the hot (−70-80° C.) agarose into it. 15 mL of agarose was pipetted into the mold to produce a 2 mm thick (z-direction) membrane for the 10 cm insert. For uniform surface distribution, 20 mL of agarose was pipetted into the insert, followed by the withdrawal of 5 mL to have 15 mL agarose.

For the 6-well insert, 2 mL of agarose was required to create the membrane. The agarose was allowed to solidify into the circular base slab for 30 minutes in the biosafety hood. The base mold enabled a tight sealing between the agarose and the insert. This seal is essential for the structural integrity of the agarose to prevent the infiltration of cells between the top and bottom chambers. The plate insert was removed from the base mold when the agarose became embedded in the extended lip of the insert. The agarose was then ready for immediate poly-D-lysine treatment or stored at 4° C. for up to 3 days for future treatment. The agarose was treated with 0.1 mg/mL poly-D-lysine (VWR, USA) to induce cationic charges that facilitate the attachment of the anionically charged cells to the agarose surface. In the biosafety hood, 3 mL poly-D-lysine was added to the surface of the agarose for 10 cm insert, while 1 mL of the solution was used for the 6-well insert. The poly-D-lysine solution was incubated on the agarose for 2 hours at room temperature. The poly-D-lysine solution was then aspirated from the agarose, followed by five washes with serum-free DMEM. The plate insert was then wrapped with parafilm and left overnight at 4° C. before the experiment started the next day. Similarly, for a control experiment to compare cell growth on the agarose to cell growth on an insert, the poly-D-lysine treated agarose layer was formed on the bottom of both a 10 cm petri dish and a 6-well plate without the plate insert.

Cell Culture Methods and Reagents

Breast cancer cell lines MDA-MB-231 (triple-negative breast cancer) were acquired from ATCC. All reagents and culture procedures used in this chapter are similar to those described in section 2.2.3. The adipose-derived stem cell donor (ASCs) was Caucasian, female, and 29 years old with a body mass index (BMI) of 32 was obtained from Obatala (New Orleans, La.).

Dynamic Co-Culture Experiments Using the 3D-Printed Plate Insert

Three cell culture experiments were performed, including both experimental and control conditions. Two single cell type control experiments were performed by either (1) directly culturing the cells on the TCP (both the 10 cm dish and the 6 well plates) or (2) adding a slab of agarose onto the bottom of the TCP (both the 10 cm dish and the 6 well plate) and then culturing the cells on top of the agarose. These control experiments were performed to confirm similar growth rates on both surfaces and to ensure the plate insert itself did not alter cell growth. Experimental co-culture conditions involved plating cells on the TCP below the insert (bottom chamber) and the agarose (top chamber). Co-culture experiments involved either the culture of a single cell type in both the top and bottom chamber (e.g., MDA-MB-231 or ASC) or the co-culture of both cell types (MDA-MB-231 in the top chamber and ASCs in the bottom chamber). The single cell line experiments were to investigate the growth rates and transcriptional changes of the cells to confirm that the cells behave the same in the insert as they do on TCP. All agarose surfaces were treated with poly-D-lysine as described above. The poly-D-lysine treated insert was removed from the 4° C. refrigerator, moved to the biosafety hood, and washed five times with the DMEM media (without serum) before the cell seeding. Next, 7.0×10⁵ cells (either 231s or ASCs) in 15 mL of media were added to the TCP plate creating the bottom chamber. The petri dish was gently agitated to facilitate the consistent distribution of cells. The plate insert was then gently placed into the dish using tweezers to ensure no air bubbles formed in the media and agarose membrane interface. If any bubbles were observed in the bottom chamber, they were aspirated. The top culture chamber was then created by adding 10 mL of media containing 7.0×10⁵ cells above the insert. Special care was taken to prevent any media spillage that would lead to cross-mixing between the top and the bottom layer. The entire dish was then rocked gently again to facilitate the uniform distribution of cells. The dish containing the plate insert was placed in the incubator at 37° C. for an hour to settle the cells before collecting ‘0-hour’ images. A similar approach was used for the control experiments using only TCP (Control-TCP) or agarose (Control-Agarose) by adding 15 mL of media containing 7.0×10⁵ cells. The cells were imaged every 24 hours for up to 96 hours using a Zeiss Primo Vert phase-contrast microscope equipped with an Axiocam 105 color digital camera. Cells were harvested at the end of the experiment using the 10 cm insert for subsequent PCR analysis. In brief, the cells were carefully scraped from the top chamber off the agarose slab using a cell scraper, with special care taken to ensure no agarose is lifted during this process. The ˜10 mL media in the top chamber with scraped cells was collected in a 15-mL tube followed by centrifugation at 300 rcf for 5 minutes. Afterward, the supernatant was discarded, and the pellet was re-suspended in 1 mL media and transferred into a 1.5 mL Eppendorf tube followed by centrifugation at 300 rcf for 5 minutes. After centrifugation, the supernatant was aspirated carefully and the pelleted was re-suspended in 50 μL of RNAlater and stored at −20° C. for RNA extraction, followed by PCR analysis. RNA extraction was conducted using Purelink RNA Mini Kit (Life Technologies, USA) via NanoDrop spectrophotometer. A similar approach was used to collect the cells from the bottom chamber after the insert was removed from the dish.

Quantification of Cell Growth in the 6-Well Plate Inserts:

These experiments followed the same setup procedure described above, with the exception of smaller volumes of media and lower numbers of cells. For this experiment, 5.0×10⁴ cells were added to the bottom chamber in 3 mL of media, while 5.0×10⁴ cells were added to the top chamber in 2 mL of media. Cell growth was assessed in triplicate using an Alamar blue assay (Invitrogen, USA) at four time points of 24, 48, 72, and 92 hours. Control experiments were also performed using TCP only and agarose only in the 6 well plates. At each time point, the insert was carefully removed from the culture chamber and placed in an adjacent empty well to allow for interrogation of cells in both the top and bottom layer. This was achieved by aspirating the media from the top chamber followed by the transfer of the insert to the empty well followed by 2 mL of media supplemented with 10% (v/v) Alamar blue. The ‘empty’ neighboring chamber had 3 mL of media in it prior to the addition of the insert to prevent the ‘top’ chamber from drying out during the Alamar blue incubation period. The bottom chamber cells were interrogated by aspirating the 3 mL growth media, followed by adding 2 mL of media containing 10% (v/v) Alamar blue. A similar procedure was performed for the TCP only, and agarose only controls. All well plates were incubated at 37° C. in the dark for four hours. The plates were analyzed using a microplate reader (Perkin Elmer Wallac Victor2, USA) with an excitation of 531 nm and emission of 595 nm. Each observation's fluorescence intensity was normalized to the baseline timepoint at 24 hours by dividing the number of the fluorescence intensity value of each time point by the 24-hour fluorescence value.

Data Analysis: Description of the Statistical Significance

The statistical differences between two groups of single culture and co-culture cells were determined by the student's t-test with a statistically significant value set at p<0.5, using OriginLab software (USA). All data presented in this study is formatted as (mean±standard deviation).

Results and Discussion

COMSOL simulations of biomolecule and oxygen mass transfer across the 3D-printed plate insert—Mass transfer diffusion studies were performed to validate the exchange of biomolecules (e.g. secreted factors by cells) and oxygen across the agarose membrane to confirm both the indirect communication between the cells in both the top and bottom chamber and also to validate that the cells were not experiencing any hypoxic conditions. The first set of simulations focused on the mass transfer of biomolecules from the bottom chamber to the top chamber. These simulations confirmed mass transfer and that a pseudo-steady-state was achieved with ˜24 hours. These simulations used an extremely high weight FITC-dextran molecule (500 kDa) to provide an upper threshold for the type of biomolecules that could be secreted. Typical growth factors are much smaller (˜5-10 kDa), while cytokines and hormones are even smaller. As such, it is anticipated that the diffusion time for these biomolecules will be much lower (˜6-10 hours). Additionally, the simulations did not account for any consumption terms for the biomolecules being processed by the top and bottom chamber cells. However, these studies were sufficient to support the claim that mass transfer did occur between the two culture chambers in the device. The second set of simulations focused on the mass transfer of oxygen into the bottom culture chamber. These simulations were important as many studies with confined chambers to culture cells suffer from reduced oxygen levels leading to hypoxia. For this simulation, the static ambient air was modeled to diffuse through the agarose membrane. The z value of 7 indicates that the diffusion begins from the agarose membrane at the height of 7 mm, which is the total distance from the bottom of the dish (z=0) to the bottom of the agarose slab. An approximate steady-state was observed at ˜8 hours, confirming that the cells in the bottom chamber would not be exposed to the hypoxic condition. Moreover, these simulations assumed that the bottom layer was completely devoid of oxygen at the start of experimentation, which is not the case. As such, these simulations support the claim that oxygen can diffuse through the agarose slab to supply the cell culture in the bottom chamber with sufficient levels for growth and proliferation. As with before, these simulations do not account for the consumption term of oxygen associated with cellular metabolism; however, reaching a pseudo steady state provides sufficient evidence that mass transfer of oxygen does indeed occur with the insert.

Experimental validation of biomolecule mass transfer across the agarose membrane in the 3D-printed plate insert—To supplement the COMSOL simulations, physical experimentation of mass transfer across the agarose membrane was performed. This was achieved by studying the accumulation of a fluorescent molecule in the top culture chamber as a function of time. For this experiment, 40 μM FITC-dextran (500 kDa) in 15 mL of dH₂O was added to the bottom chamber in a 10 cm petri dish. The assembled insert was then placed in the dish followed by the addition of 10 mL of dH₂O into the top chamber. Five 10 μL samples were collected at five distinct locations in the top culture chamber at nine time points (0, 1, 2, 4, 8, 12, 24, 48, and 72 hours). The relative amount of FITC in the top chamber was determined using a NanoDrop. The data demonstrated that the amount of FITC-dextran in the top chamber increased as a function of time following a logarithmic trend until a stable concentration of ˜40 μM was achieved ˜24 hours (See FIG. 5B). These results align with the COMSOL simulations and confirm that biomolecules can diffuse across the agarose membrane in the plate insert to support the indirect communication between two different cell types cultured on two layers.

Qualitative assessment of MDA-MB-231 cell morphology and viability in the 3D-printed plate insert—It was necessary to confirm that cells grew and behaved the same in the 3D-printed plate insert prior to co-culture experimentation. To achieve this, one experimental and two control conditions were performed using MDA-MB-231 cells. The two control conditions consisted of cells plated directly on the surface of the TCP dish or directly on the surface of agarose that had been layer on the bottom of the TCP dish. The experimental condition consisted of the assembled plate insert where cells were plated on the TCP in the bottom chamber and on the agarose in the top chamber. Images of the cells were collected under each condition to evaluate and compare cellular morphology and approximate changes in cell number. The cells all exhibited the same mesenchymal morphology associated with triple-negative breast cancer cells in the TCP control, the agarose control, and in both chambers of the experimental plate insert experiment. These results are similar to those observed when culturing 231 cells on agarose in a microfluidic device. This finding is also important because any observed changes in cellular behavior (e.g., growth, morphology, genotype) should be due to the presence of a second cell type and not the 3D-printed plate insert. These images also support the notion that the cells were growing in the plates by a relative increase in the number of cells. This qualitative assessment was supplemented with quantitative growth rate studies using Alamar blue (see below). Some efforts were taken to manually count the cells in both the control and experimental conditions to provide some insight into the respective growth rates of the cells. This manual counting approach was both tedious and inconsistent due to human error. As such, the manually counting of cells was abandoned in favor of the Alamar blue approach. In addition to studying cell morphology, a terminal viability stain was performed to confirm that the cells were still alive after 72 hours of culture in the insert. As seen in FIG. 9, the majority of the cells cultured in the plate insert were alive after 72 hours. This result was the same in both the TCP and agarose control experiments. This confirmed that the plate insert did not alter cell viability. In addition to studying cell behavior in the insert, the preliminary studies allowed for the harvesting of cells to evaluate the RNA yields for post-experimental analysis via PCR. The RNA extraction purity is measured by the absorption ratio of the wavelength of 260/280 and 260/230. A ratio of ˜1.8 is considered pure for DNA, while ˜2.0 is pure for RNA. The ratio of 260/230 is used as a secondary measure of nucleic acid purity at the ratio of ˜2.0. Preliminary extraction of

RNA qualified the primary purity test while the secondary purity test needs to improve (Table 1).

TABLE 1
RNA Extraction from 10 cm petri dishes and
plate inserts after harvesting of cells

| | Culture | | RNA Extraction | | |
|---|---|---|---|---|---|
| Cell line | medium | Replication | 260/280 | 260/230 | Yield(ng/mL) |
| MDA- | TCP (Control) | 1 | 2.08 | 1.62 | 1107.4 |
| MB-231 | | 2 | 2.08 | 1.41 | 244.0 |
| | | 3 | 2.08 | 1.34 | 276.9 |
| | Agarose | 1 | 2.08 | 0.78 | 364.0 |
| | (Control) | | | | |
| | | 2 | 2.08 | 1.58 | 230.5 |
| | | 3 | 2.07 | 1.99 | 445.7 |
| | Insert-Agarose | 1 | 2.06 | 1.69 | 123.9 |
| | | 2 | 2.04 | 1.53 | 246.5 |
| | | 3 | 2.05 | 2.19 | 293.6 |
| ASC | Insert-Bottom | 1 | 2.04 | 1.16 | 466.0 |
| | TCP | | | | |
| | | 2 | 2.03 | 2.09 | 327.5 |
| | | 3 | 2.05 | 1.33 | 401.7 |

The RNA yield was also higher in all instances than the required yield of 100 ng/mL.

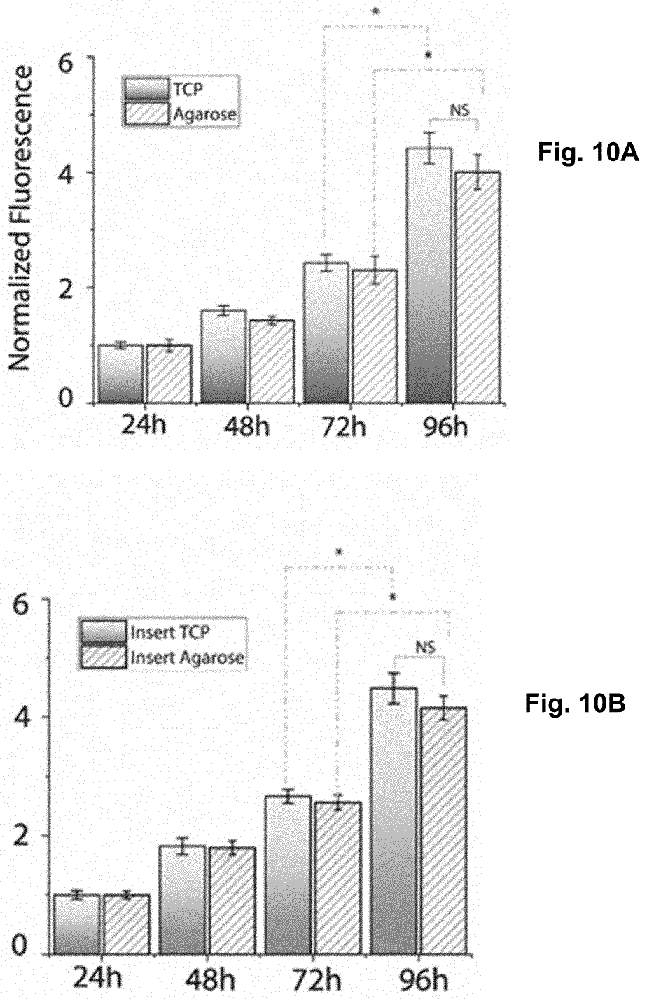

Quantitative assessment of cell growth in the 3D-printed plate insert—The data presented in the previous section suggested the cells behave the same in the plate insert as in traditional TCP dishes. However, a more quantitative study was needed to confirm similar growth rates across both experimental and control conditions. To accomplish this, a smaller version of the plate insert was designed and fabricated to fit into a 6-well plate. Typical approaches to directly quantify cellular viability and growth utilize fluorescent stains coupled with well plates and a fluorometric plate reader. Alamar blue is one of these stains that functions as a cell health indicator by reduced resazurin (a blue dye) to resorufin (a red dye) in intact, healthy cells. The reduced resorufin is highly fluorescent and proportional to the number of living cells that respire. It is highly sensitive and provides direct information on both cell viability and number growth. In terms of cell growth, the fluorescent signal generated by the Alamar blue assay can be normalized against a baseline value (e.g., the fluorescent signal at 24 hours) to quantify cell growth directly. To investigate cell growth in the 3D-printed plate insert, both experimental and control conditions were repeated in the 6-well plates similar to as described above. Four separate 6-well plates were used for 24-, 48-, 72-, and 96-hour time points because the Alamar blue assay is a terminal assay. Each condition was performed in triplicate to allow for statistical analysis with the average fluorescent signals for each time normalized against the fluorescent signal for the 24-hour time to allow for a direct comparison. As can be seen in FIGS. 10A-10B, there was a statistically significant (p-value <0.05) difference between the fluorescent signal for the 72 hour and 96 hour time points in both the control (TCP and agarose) and experimental (plate insert top and bottom chambers) confirming an increase in cells due to growth. Moreover, there was no significant difference in the fluorescent signal between cell cultures on TCP versus agarose (FIG. 10A) or between the top and bottom chamber in the plate insert (FIG. 10B). A similar statistical comparison was performed between the 72- and 96-hour signals for the TCP control, agarose control, insert bottom layer, and insert top layer which was also found to be not significant. These findings provide quantitative data that the growth rates for MDA-MB-231 cells were the same in both the plate insert and the control plates. Moreover, the relatively small error bars in FIGS. 10A-10B support the claim of uniform plating and performance of the plate inserts across all well of the 6-well plate. Finally, the growth rates observed for the MDA-MB-231 cells in the plate insert are similar to those previously found using a microfluidic approach. These findings confirm that the plate insert and the agarose do not alter cellular behavior which supports its use for dynamic co-culture applications between two different cell types.

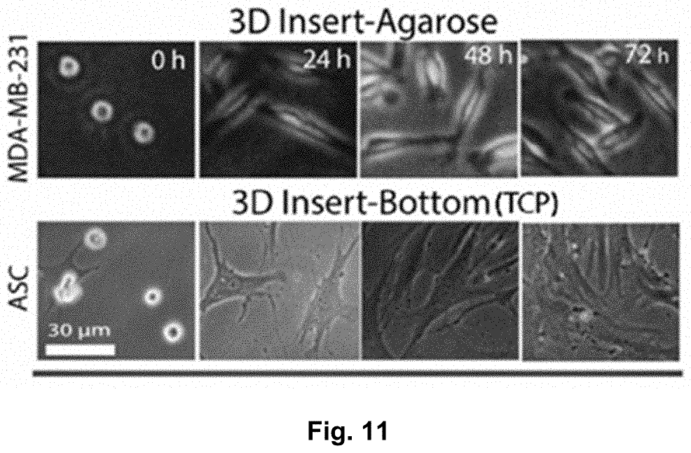

Dynamic co-culture of triple-negative breast cancers with adipose-derived stem cells (ASCs)—Once it was confirmed that the plate insert could support cell growth with no changes in cellular behavior, the final test was to perform dynamic co-culture studies between two cell types. Our prior work demonstrated that the co-culture of MDA-MB-231 triple-negative breast cancer cells with ASCs results in increase growth rates and drug resistance (Rahman et al, 2020). Based on this, we wanted to confirm that both cell types could be cultured using the plate insert. The first experiment performed was a single culture experiment with the ASCs to confirm that their morphology (and indirectly behavior) was not altered due to being cultured on agarose and in the insert. A similar approach was applied here as described above using both TCP and agarose controls and a plate insert experimental condition (FIG. 7A-7C). These representative images confirmed a similar morphology for the ASCs in all three conditions confirming that culturing the ASCs in the plate insert did not alter cellular morphology. Finally, a co-culture experiment was performed by culturing the ASCs in the bottom chamber and the MDA-MB-231 cells in the top chamber (FIG. 11). The qualitative imaging experiments support the ability to co-culture both cell types using the plate insert. Moreover, a cursory evaluation of the cells suggests that the cells are indeed growing during the 72-hour experiment, as evidenced by a greater number of cells in the 72-hour time point. At the end of the experiment, both MDA-MB-231 and ASCs were harvested, and RNA was extracted for subsequent analysis by PCR to identify changes in the cellular genotype. As we found with the 231 only control, we were able to obtain sufficient amounts and purities of the RNA for both cell types (Table 1). These findings support the use of the 3D-printed plate insert as a novel approach for the dynamic co-culture of two different cell types. Moreover, additional studies are underway repeating the quantitative cell growth experiment using Alamar blue to investigate if the presence of ASCs enhances the growth rate of 231 cells and vice versa. Additionally, all of the samples collected thus far are currently being processed and run by PCR to provide a baseline and experimental transcriptional change (231 only or ASC only) to identify specific changes that are up/down-regulated due to the presence of another cell types. These studies will provide new insight into the role that stromal cells play in cancer cell progression and drug resistance.

### Results

The technology fills a gap in current co-culture approaches in that it provides sufficient cellular yields for traditional biochemical interrogation of cells (e.g., PCR and Western Blot) coupled with the ability to visualize the cells during experimentation continuously. The 3D-printed plate insert is superior to the Transwell assay in many ways, including its ability to harvest cells for downstream analysis, its ability to visualize cells during experimentation, and its ability to prevent cellular invasion from the top chamber into the bottom chamber. The dimensions of two different inserts were optimized to fit in either a 10 cm petri dish or a 6-well plate. Mass transfer (both biomolecules and oxygen) was validated computationally and experimentally to confirm the exchange of paracrine signals between the cell types and that they were not experiencing hypoxia. A series of control experiments were performed to confirm there was no difference in cellular morphology, cellular growth, or cellular viability between the plate insert and TCP/agarose controls. Finally, a proof-of-concept study was performed to demonstrate the ability to co-culture triple-negative breast cancer cells (MDA-MB-231) and ASCs.

### Example 2 References

[1] S. M. Rahman, J. M. Campbell, R. N Coates, K. M. Render, C. E. Byrne, E. C. Martin, and A. T. Melvin. “Evaluation of intercellular communication between breast cancer cells and adipose-derived stem cells via passive diffusion in a two-layer microfluidic device.” Lab on a Chip 20:2009 (2020)

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, “about 0” can refer to 0, 0.001, 0.01, or 0.1. In an embodiment, the term “about” can include traditional rounding according to significant figures of the numerical value. In addition, the phrase “about ‘x’ to ‘y’” includes “about ‘x’ to about ‘y’”.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

What is claimed is:

1. A cell plate insert, comprising an interior lip and legs on a bottom side, and having a diameter to provide a snug fit within a cell plate or well.

2. The cell plate insert of claim 1, further comprising a cell support membrane disposed on an interior lip of the cell plate insert.

3. The cell plate insert of claim 1, wherein the cell support membrane comprises agarose.

4. The cell plate insert of claim 1, wherein the legs are of a height sufficient to provide an equal distance between a first tissue culture substrate disposed on a bottom of the cell plate and the cell culture support media.

5. The cell plate insert of claim 1, wherein the cell plate is a petri dish having a 10 cm diameter and wherein the diameter of the cell plate insert is about 95 mm to 99 mm.

6. The cell plate insert of claim 5, wherein a height of the cell plate insert is about 10 mm including the legs, and wherein the legs have a height of about 2 mm.

7. The cell plate insert of claim 1, wherein the cell plate is a multi-well plate having wells of about 35 mm diameter and wherein the diameter of the cell plate insert is about 30 mm to 34 mm.

8. The cell plate insert of claim 7, wherein a height of the cell plate insert is about 10 mm to 10.5 mm excluding the legs, and wherein the legs have a height of about 2 mm to 3 mm.

9. A system for cell co-culture, comprising a cell plate insert having an interior lip on a top side and legs on a bottom side; and
wherein the cell plate insert has a diameter to provide a snug fit within a cell plate.

10. The system for cell co-culture of claim 9, further comprising a cell support membrane disposed on an interior lip of the cell plate insert such that the cell plate insert forms a top chamber and a bottom chamber.

11. The system for cell co-culture of claim 9, further comprising a cell plate comprising a first tissue culture substrate, wherein the cell plate insert is inserted on top of the tissue culture substrate.

12. The system for cell co-culture of claim 9, wherein in the tissue culture substrate comprises tissue culture plastic.

13. The system for cell co-culture of claim 9, wherein the cell support membrane comprises agarose.

14. The system for cell co-culture of claim 9, further comprising a second tissue culture substrate disposed on top of the cell support membrane.

15. The system for cell co-culture of claim 9, further comprising a cell plate lid.

16. The system for cell co-culture of claim 9, further comprising a cell support membrane formation base having a diameter equivalent to a diameter of the interior lip.

17. The system for cell co-culture of claim 9, wherein the cell plate insert is optically transparent.

18. The system for cell co-culture of claim 14, wherein the cell plate insert allows for oxygen permeability.

19. The system for cell co-culture of claim 14, wherein cells cultured in the top chamber do not infiltrate cells cultured in the bottom chamber.
